(19) United States
(12) Patent Application Publication
(10) Pub. No.: US 2021/0220423 A1
(43) Pub. Date: Jul. 22, 2021

(54) PAIN RELIEVER COMPOSITION

(71) Applicant: Earl Marek, Hilton Beach (CA)

(72) Inventor: Earl Marek, Hilton Beach (CA)

(21) Appl. No.: 17/223,308

(22) Filed: Apr. 6, 2021

(51) Int. Cl. A61K 36/886; A61K 36/534; A61K 36/87; A61K 36/752; A61K 36/54; A61K 9/00

(52) U.S. Cl. CPC A61K 36/886; A61K 36/534; A61K 9/0014; A61K 36/752; A61K 36/54; A61K 36/87

(57) ABSTRACT

A topical pain reliever composition that includes a blend of peppermint essential oil, rosemary essential oil, aloe vera oil, grapeseed oil, avocado oil, camphor essential oil, lemon essential oil, tangerine essential oil, camphor distillate, lavender essential oil, and tea tree essential oil.

### FIELD OF INVENTION

The present invention relates to a pain reliever composition, and more particularly, the present invention relates to a composition having natural ingredients for bodily pain relief in humans.

### BACKGROUND

A variety of pain relievers are known in the art for use in mild pain, moderate pain, and severe pain. The known pain relievers can be classified based on their chemical class, mechanism of action, or use. Also, known as analgesics or pain killers, these belong to different chemical classes and have different mechanisms of action. A single medication or a combination can be prescribed based on the severity of the pain. Most used pain relievers belong to the class of NSAIDs i.e., non-steroidal anti-inflammatory agents. Many of the NSAIDs are available as OTC drugs i.e., they can be self-prescribed and do not require a prescription from a physician.

Although a range of pain relievers are known for a variety of pains, most of the known pain relievers are synthetic compounds and have many side-effects. Long-term use of pain relievers is not advised because of the side-effects. Well-known NSAIDs have common side effects, such as stomach upset and kidney damage.

Moreover, pain is generally described as a disabling disorder that prevents a person from living a normal life. Recurrent pain conditions also affect the professional life of a person and people get frustrated when physical ailments prevent them from operating near peak efficiencies. For example, many people experience the disabling condition of fibromyalgia syndrome (“FMS”), consisting of widespread muscle pain and stiffness and women experience significant amounts of discomfort at selected times during their menstrual cycles, a condition often identified as premenstrual syndrome (“PMS”). Conventional pain relievers such as aspirin and acetaminophen have produced only moderate success in addressing the physical symptoms associated with FMS, PMS, and other such disorders. It is frequently necessary for people who suffer from significant discomfort levels to obtain treatment from a physician who often must prescribe stronger medications to reduce the discomfort to an acceptable level. A drawback associated with the use of such stronger medications is that these types of medications often negatively affect the patient's mental and/or physical acuity.

Thus, a long-unsolved desire is there for a composition having natural ingredients for relieving bodily pains.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to pain reliever composition that has all-natural ingredients.

It is another object of the present invention that the pain reliever composition has insignificant or no side-effects.

It is a further object of the present invention that the pain reliever composition can be used for a range of bodily pains.

It is an additional object of the present invention that the pain reliever composition can be easily applied to the affected bodily area.

It is still an additional object of the present invention that the pain reliever composition can provide instant relief.

It is still a further object of the present invention that the composition can provide relief in pain associated with fibromyalgia syndrome.

It is yet a further object of the present invention that the composition can provide relief in pain associated with premenstrual syndrome.

It is an object of the present invention that the composition can be used topically for providing relief in bodily pains.

In one aspect, disclosed is a topical pain reliever composition comprising peppermint essential oil, rosemary essential oil, aloe vera oil, grapeseed oil, avocado oil, camphor essential oil, lemon essential oil, tangerine essential oil, camphor distillate, lavender essential oil, and tea tree essential oil.

In one aspect, disclosed is a method of preparing a pain reliever composition from a mixture of peppermint essential oil, rosemary essential oil, aloe vera oil, grapeseed oil, avocado oil, camphor essential oil, lemon essential oil, tangerine essential oil, camphor distillate, lavender essential oil, and tea tree essential oil.

In one aspect, disclosed is a method of providing relief in bodily pains by topically applying a pain reliever composition comprising peppermint essential oil, rosemary essential oil, aloe vera oil, grapeseed oil, avocado oil, camphor essential oil, lemon essential oil, tangerine essential oil, camphor distillate, lavender essential oil, and tea tree essential oil to an affected body part or area.

In one aspect, the pain reliever composition can be applied to the abdomen for relief in pain associated with premenstrual syndrome.

In one aspect, the pain reliever composition can provide relief in pain associated with fibromyalgia syndrome.

These and other objects and advantages of the embodiments herein and the summary will become readily apparent from the following detailed description.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as compositions or methods of treatment. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Unless otherwise indicated, all numbers expressing quantities of ingredients used in this disclosure are to be understood as being modified in all instances by the term “about.” Accordingly, unless indicated to the contrary, the numerical parameters set forth in this disclosure are approximations that may vary depending upon the desired properties sought to be obtained by the present disclosure. At the very least, and not as an attempt to limit the application of the doctrine of equivalents to the scope of any claims, each numerical parameter should be construed in light of the number of significant digits and ordinary rounding approaches.

“Pharmaceutical composition” or “compositions” hereinafter refers to preparations which are in such a form as to permit the biological activity of the active agents to be unequivocally effective, and which contain no additional components which are toxic as administered to the patients.

Disclosed is a composition that can provide relief in bodily pains when applied topically to the human body proximate to the location of the pain. For example, the pain reliever composition of the present invention is particularly suited for relieving pain associated with premenstrual syndrome wherein the pain reliever composition is applied to the person's abdomen. The pain reliever composition of the present invention can be used in relieving pain associated with other physical ailments, such as muscle strains, muscle sprains, muscle aches, bruises, arthritis, and joint pain. Additionally, the pain reliever composition reduces swelling when applied topically in areas of a body having swelling. The disclosed pain reliever composition is preferably found suitable in providing relief in pain associated with fibromyalgia syndrome and premenstrual pains. An advantage of the disclosed composition is that it can be made from organically derived essential oils, which have no significant side effects.

In one exemplary embodiment, the disclosed pain reliever composition includes peppermint essential oil, rosemary essential oil, aloe vera oil, grapeseed oil, avocado oil, camphor essential oil, lemon essential oil, tangerine essential oil, camphor distillate, lavender essential oil, and tea tree essential oil. The concentration of peppermint essential oils can be in the range of about 48 to 73 percent by weight, and preferably about 54 percent by weight. The concentration of rosemary essential oil can be in the range of about 15 to 25 percent by weight, and preferably about 23 percent. The concentration of aloe vera oil can be in the range of about 5 to 10 percent by weight, and preferably about 8 percent by weight. The concentration of grapeseed oil can be in the range of about 3 to 8 percent by weight, and preferably about 4 percent by weight. The concentration of avocado oil can be in the range of about 3 to 8 percent by weight, and preferably about 4 percent by weight. The concentration of camphor essential oil can be in the range of about 2 to 8 percent by weight, and preferably about 3 percent by weight. The concentration of lemon essential oil can be in the range of about 1 to 8 percent by weight, and preferably about 2 percent by weight. The concentration of tangerine essential can be in the range of about 1 to 8 percent by weight, and preferably about 2 percent by weight. The concentration of camphor distillate can be in the range of about 1 to 3 percent by weight, and preferably about 1 percent by weight. The concentration of lavender essential oil can be in the range of about 1 to 8 percent by weight, and preferably about 1 percent by weight. The concentration of tea tree essential oil can be in the range of about 1 to 8 percent by weight, and preferably about 1 percent by weight.

The Peppermint essential oil can be obtained from Mentha arvensis using the whole herb, twig flowerings, herb flowerings, and leaves. The Rosemary essential oil can be obtained from Rosmarinus officinalis using flowers, leaves, and herb top flowerings. Aloe vera oil can include organic aloe vera leaf powder, olive oil, and vitamin E oil. The Grapeseed oil can be obtained from Vitis vinifera. Avocado oil can be obtained from Persea gratissima. Camphor essential oil can be obtained from Cinnamomum camphora using wood. The Lemon essential oil can be obtained from Citrus limon derived from fruit peel. The Tangerine essential oil can be obtained from Citrus reticulate blanca var tangerine using fruit peel. Camphor (isolate) can include d-camphor. The lavender essential oil can be obtained from Lavandula angustifolia using herb top flowerings. Tea tree essential oil can be obtained from Melaleuca alternifolia using leaves. The disclosed composition can be obtained by blending the above essential oils. The composition can be stored in a cool and dark environment.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for providing pain relief in bodily pains, the method comprising the steps of:
providing a pain reliever composition comprising peppermint essential oil, rosemary essential oil, aloe vera oil, grapeseed oil, avocado oil, camphor essential oil, lemon essential oil, tangerine essential oil, camphor distillate, lavender essential oil, and tea tree essential oil; and
topically applying the pain relief composition to an affected area.

2. The method according to claim 1, wherein the bodily pains are associated with premenstrual pains and the pain relief composition is applied to abdomen.

3. The method according to claim 1, wherein the bodily pains are associated with fibromyalgia syndrome.

4. The method according to claim 1, wherein the bodily pains are associated with arthritis and the pain relief composition is applied to joints.

5. The method according to claim 1, wherein the peppermint essential oil is in the range of about 48 to 73 percent by weight, the rosemary essential oil is in the range of about 15 to 25 percent by weight, the aloe vera oil is in the range of about 5 to 10 percent by weight, the grapeseed oil is in the range of about 3 to 8 percent by weight, the avocado oil is in the range of about 3 to 8 percent by weight, the camphor essential oil is in the range of about 2 to 8 percent by weight, the lemon essential oil is in the range of about 1 to 8 percent by weight, the tangerine essential is in the range of about 1 to 8 percent by weight, the camphor distillate is in the range of about 1 to 3 percent by weight, the lavender essential oil is in the range of about 1 to 8 percent by weight, and the tea tree essential oil is in the range of about 1 to 8 percent by weight.

6. The method according to claim 1, wherein the peppermint essential oil is about 54 percent by weight, the rosemary essential oil is about 23 percent, the aloe vera oil is about 8 percent by weight, the grapeseed oil is about 4 percent by weight, the avocado oil is about 4 percent by weight, the camphor essential oil is about 3 percent by weight, the lemon essential oil is about 2 percent by weight, the tangerine essential is about 2 percent by weight, the camphor distillate is about 1 percent by weight, the lavender essential oil is about 1 percent by weight, and the tea tree essential oil is about 1 percent by weight.

7. A pain reliever composition comprising peppermint essential oil, rosemary essential oil, aloe vera oil, grapeseed oil, avocado oil, camphor essential oil, lemon essential oil, tangerine essential oil, camphor distillate, lavender essential oil, and tea tree essential oil.

8. The pain reliever composition according to claim 7, wherein the peppermint essential oil is in the range of about 48 to 73 percent by weight, the rosemary essential oil is in the range of about 15 to 25 percent by weight, the aloe vera oil is in the range of about 5 to 10 percent by weight, the grapeseed oil is in the range of about 3 to 8 percent by weight, the avocado oil is in the range of about 3 to 8 percent by weight, the camphor essential oil is in the range of about 2 to 8 percent by weight, the lemon essential oil is in the range of about 1 to 8 percent by weight, the tangerine essential is in the range of about 1 to 8 percent by weight, the camphor distillate is in the range of about 1 to 3 percent by weight, the lavender essential oil is in the range of about 1 to 8 percent by weight, and the tea tree essential oil is in the range of about 1 to 8 percent by weight.

9. The pain relief composition according to claim 7, wherein the peppermint essential oil is about 54 percent by weight, the rosemary essential oil is about 23 percent, the aloe vera oil is about 8 percent by weight, the grapeseed oil is about 4 percent by weight, the avocado oil is about 4 percent by weight, the camphor essential oil is about 3 percent by weight, the lemon essential oil is about 2 percent by weight, the tangerine essential is about 2 percent by weight, the camphor distillate is about 1 percent by weight, the lavender essential oil is about 1 percent by weight, and the tea tree essential oil is about 1 percent by weight.

10. A method of preparing a pain reliever composition, the method comprising the steps of mixing predetermined amounts of peppermint essential oil, rosemary essential oil, aloe vera oil, grapeseed oil, avocado oil, camphor essential oil, lemon essential oil, tangerine essential oil, camphor distillate, lavender essential oil, and tea tree essential oil.

11. The method according to claim 10, wherein the peppermint essential oil is in the range of about 48 to 73 percent by weight, the rosemary essential oil is in the range of about 15 to 25 percent by weight, the aloe vera oil is in the range of about 5 to 10 percent by weight, the grapeseed oil is in the range of about 3 to 8 percent by weight, the avocado oil is in the range of about 3 to 8 percent by weight, the camphor essential oil is in the range of about 2 to 8 percent by weight, the lemon essential oil is in the range of about 1 to 8 percent by weight, the tangerine essential is in the range of about 1 to 8 percent by weight, the camphor distillate is in the range of about 1 to 3 percent by weight, the lavender essential oil is in the range of about 1 to 8 percent by weight, and the tea tree essential oil is in the range of about 1 to 8 percent by weight.

12. The method according to claim 10, wherein the peppermint essential oil is about 54 percent by weight, the rosemary essential oil is about 23 percent, the aloe vera oil is about 8 percent by weight, the grapeseed oil is about 4 percent by weight, the avocado oil is about 4 percent by weight, the camphor essential oil is about 3 percent by weight, the lemon essential oil is about 2 percent by weight, the tangerine essential is about 2 percent by weight, the camphor distillate is about 1 percent by weight, the lavender essential oil is about 1 percent by weight, and the tea tree essential oil is about 1 percent by weight.

13. The method according to claim 1, wherein the bodily pains are associated with muscle strains, muscle sprains, or muscle aches.

14. The method according to claim 1, wherein the bodily pains are associated with bruises.

15. The method according to claim 1, wherein the camphor distillate comprises d-camphor.
